(19) United States
(12) Patent Application Publication
(10) Pub. No.: US 2023/0372079 A1
(43) Pub. Date: Nov. 23, 2023

(54) VASCULAR STENT-GRAFT

(71) Applicant: ASCENSE MEDICAL GmbH, Vienna (AT)

(72) Inventors: Michael MOORE, Surfside, FL (US); Roman GOTTARDI, Vienna (AT)

(21) Appl. No.: 18/248,606

(22) PCT Filed: Oct. 12, 2021
(86) PCT No.: PCT/IB2021/059330

§ 371 Date: Apr. 11, 2023

(30) Foreign Application Priority Data
Oct. 12, 2020 (ZA) 2020/06308

(51) Int. Cl. A61F 2/07

(52) U.S. Cl. CPC A61F 2/07; A61F 2250/0039; A61F 2250/0037; A61F 2002/072

(57) ABSTRACT

Disclosed is a vascular stent-graft which includes a tubular body having a tubular wall which is comprised of at least a first layer of a fabric, said body including a proximal anchor section of a cylindrical form, preformed with a first diameter and a first length, a distal treatment section of a tapered form and having a second length, and a transition section which tapers the tubular body from the anchor segment to the treatment segment, at least one stent of a first set engaged with the anchor section, adapted to bias the anchor section from the first diameter to a second diameter which exceeds the first diameter.

### BACKGROUND OF THE INVENTION

This invention relates to a vascular stent graft for use in the treatment of an aortic dissection.

Current thoracic aortic stent grafts are primarily designed for the treatment of descending thoracic aortic aneurysms by providing a generally tubular body which acts as an artificial blood vessel to exclude the aneurysm. The function of a stent graft in the treatment of this disease dictates its form.

The tubular graft body, typically, is made of a fabric of a suitable plastics material such as polyethylene terephthalate (PET) or spun fibres of polytetrafluoroethylene (PTFE), which extends between a proximal end and a distal end. A plurality of circumferentially expansive stent elements engaged (stitched or adhered) to a wall of the tubular body, spaced at longitudinal intervals.

Each stent element typically is a nitinol wire ring which is wave-formed to allow for circumferential expansion and compression, about the wave sections. This configuration allows for each stent element to circumferentially expand within the aorta, once appropriately located, from a circumferentially contracted state during insertion. Problematically, with the tubular graft body and each stent element being oversized, relatively to the diameter of the aorta into which it is to be deployed, there is a significant radial force imparted on the aorta and an infolding or pleating of the tubular graft body when the graft body is implanted.

A typical stent-graft has a proximal and a distal anchor segment, ending at the proximal and distal ends respectively, which are adapted (by the number, size, configuration and/or position of the stent elements in these segments) to anchor in healthy aortic tissue proximal (hereinafter “proximal landing zone”) and distal (hereinafter “distal landing zone”) to the aneurysm. Such placement allows the stent graft to form a seal within the aorta, with the medial portion of the stent graft bridging the aneurysm and excluding it from the circulating blood flow.

To achieve a good seal, an oversizing of the stent-graft relative to the aortic diameter at the proximal and the distal landing zones is required. For aneurysms, an oversizing factor of approximately 20% is chosen.

These stent-grafts also are being used in treating aortic dissections. Problems with such use arise from the fact that this disease is physiologically very different to an aneurysm. A dissection is caused by a tear in the intimal layer of the aorta, allowing blood to leak into, and flow along between the aortic wall layers, creating a “false lumen” which is separate from the “true lumen”. A dissection can be confined to the thoracic aorta but can also extend down to the abdominal aorta.

The main goal of endovascular treatment of an aortic dissection with a stent-graft is to occlude the entry tear which allows the blood to flow into the aortic wall, between the aortic wall layers. To achieve effective occlusion, the stent-graft does not have to cover the whole dissection but only the most proximal entry tears.

The stent-grafts described above are stiff, both radially and longitudinally, due to both the radial load on the woven fabric material of the body, caused by the oversize dimensions of the stent elements, and the composition of the fabric itself. Consequently, the stent-grafts impose high radial forces on the aorta, at least within the aortic landing zones.

These ridged design attributes reduce the “windkessel function” of the aorta and can induce left ventricular hypertrophy, increase blood pulse-wave velocity, and consequently induce arterial hypertension.

Also, mechanical complications can be linked to this lack of compliance and resulting in compliance mismatch. These complications are: retrograde aortic dissection, which propagates proximally; or distal stent-graft induced new entries (dSINE). The former condition requires open surgery, the latter either open surgery or secondary stent graft placement.

The present invention at least partially addresses the aforementioned problem.

### SUMMARY OF INVENTION

Hereinafter, the terms “proximal” and “distal” refer to proximity relatively to the arch of the aorta.

Hereinafter, the term “knitted” refers to a fabric construction whereby a single yarn or thread is interloped or interlaced as opposed to “woven” whereby the fabric is created using several warps, or longitudinal yarns, and wefts, or latitudinal yarns.

Hereinafter, the term “preformed” refers to a forming step made on the tubular body prior to a stent or stents being engaged with said body.

Hereinafter, when used to describe a structure, the term “compliance” refers to the ability of the structure to expand from a native state and then to recoil back to the native state.

In a first aspect, the invention provides a stent-graft for treating a dissection in the descending aorta, the stent-graft including:
a tubular body having a tubular wall;
a plurality of stents engaged with the tubular wall;
wherein the tubular wall includes an first layer of a fabric, and a second layer comprised of a liquid-tight material; and
wherein the tubular wall is radially compliant within a range 5 to 20%.

The fabric may be a knitted or a woven fabric.

The knitted or woven fabric may comprise yarns of at least a polyester material such as, for example, a knitted polyethylene terephthalate (PET) or the like.

The liquid-tight material may be an elastomeric polymeric material such as, for example, polyurethane or the like.

The elastomeric polymeric material of the second layer may be provided in a solid preform, such as a sheet or a tube, which covers, binds to or encapsulates the first layer. Alternatively, the elastomeric polymeric material of the second layer may be applied, by any suitable means, to the first layer in liquid form and which then solidifies thereon, coating or encapsulating the first layer.

The plurality of stents may be fixed to tubular wall, sandwiched or interposed between the first layer and the second layer. Alternatively, the plurality of stents may be fixed to tubular wall, embedded within the second layer.

In a second aspect, the invention provides a stent-graft for treating a dissection in the descending aorta, the stent graft including:
a tubular body having a tubular wall which is comprised of at least a first layer of a fabric, said body including:
a proximal anchor section of a cylindrical form, preformed with a first diameter and a first length;
a distal treatment section of a tapered form and having a second length; and
a transition section which tapers the tubular body from the anchor segment to the treatment segment;
at least one stent of a first set engaged with the anchor section, adapted to bias the anchor section from the first diameter to a second diameter which exceeds the first diameter; and
a plurality of stents of a second set engaged with the treatment section, each adapted to maintain the tapered form.

Preferably, the first diameter is sized to approximate the aortic diameter at the proximal landing zone.

The first diameter may be in a range 18 mm to 40 mm.

The second diameter may exceed the first diameter by about 5%, about 6%, about 7%, about 8%, about 9%, about 10%, about 11%, about 12%, about 13%, about 14%, about 15%, about 16%, about 17%, about 18%, about 19%, about 20% or any range in between. Preferably, the second diameter exceeds the first diameter between 9%-11%.

The treatment section may taper from a third diameter adjacent the transition section to a fourth diameter at the distal end.

The third diameter may range from 17 mm to 39 mm. The fourth diameter may range from 15 mm to 38 mm respectively.

The first length may be in a range 20 mm to 50 mm.

The transition section length may be in a range 1 mm to 10 mm.

The second length may be in a range 50 to 250 mm.

The fabric may be adapted to allow for radial compliance of the tubular wall by about 5%, about 6%, about 7%, about 8%, about 9%, about 10%, about 11%, about 12%, about 13%, about 14%, about 15%, about 16%, about 17%, about 18%, about 19%, about 20% or any range in between.

The fabric may be a knitted or a woven fabric.

The knitted or woven fabric may comprise yarns of at least a polyester material such as, for example, polyethylene terephthalate (PET) or the like.

Preferably, the knitted or woven fabric includes a combination of elastomeric and non-elastomeric yarns.

The tubular wall may include a second layer which covers, coats or encapsulates the first layer.

The second layer may comprise of a liquid-tight material. The liquid-tight material may be, for example, polyurethane or the like.

The liquid-tight material may be provided in a solid preform, such as a sheet or a tube, which covers and binds to the first layer. Alternatively, the liquid-tight material may be a liquid which is applied, by any suitable means, to the first layer and which then hardens thereon.

The stents of the first set and the second set may be fixed to the anchor section and the treatment section respectively, sandwiched or interposed between the first layer and the second layer. Alternatively, the stents of the first set and the second set may be fixed to the anchor section and the treatment section respectively, embedded within the second layer.

Each stent of the first set and the second set may be a circumferential self-expanding stent.

Each stent of the first and the second set may be made of a suitable super-elastic alloy such as, for example, nitinol.

Each stent of the first set and the second set may have a waveform wire body configuration or an expanded slotted tube configuration.

The anchor section may have a plurality of stents of the first set engaged therewith, preferably two.

Each stent of the second set may be engaged with the treatment section sequentially, at intervals, along the second length. Preferably the intervals are regular. More preferably, the interval is between 13 to 15 mm.

Each stent of the first set may be adapted to maintain the second diameter against an elastic recoil force imposed by the aorta on the anchor section.

Each stent of the second set may be adapted to maintain the tapered form of the treatment segment, in use of the stent-graft, without a radial force or with a negligible radial force being imposed on the aorta, when a true lumen of the aorta returns to a native pre-dissected diameter.

The anchor section may have a fenestration or may be formed with a side branch which is in fluid communication with the lumen.

### BRIEF DESCRIPTION OF THE DRAWINGS

The invention is further described by way of examples with reference to the accompanying drawings in which:

FIG. 1 diagrammatically represents types of aortic dissections;

FIG. 2 is a view in elevation of a stent-graft in accordance with the invention;

FIG. 3 is an isometric view of the stent-graft of FIG. 2;

FIG. 4 is a view in elevation of a stent-graft in accordance with a second embodiment of the invention;

FIG. 5 is an isometric view of the stent-graft of FIG. 2 or FIG. 4, showing a fenestration;

FIG. 6 is an isometric view of a stent of the stent-graph;

FIG. 7 is a view in elevation of the stent of FIG. 6;

FIG. 8 is a view in cross-section through the wall of the tubular body of the stent graph;

FIG. 9 diagrammatically illustrates the stent-graft deployed within the descending aorta in a first position; and

FIG. 10 diagrammatically illustrates the stent-graft deployed within the descending aorta in a second position.

### DESCRIPTION OF PREFERRED EMBODIMENTS

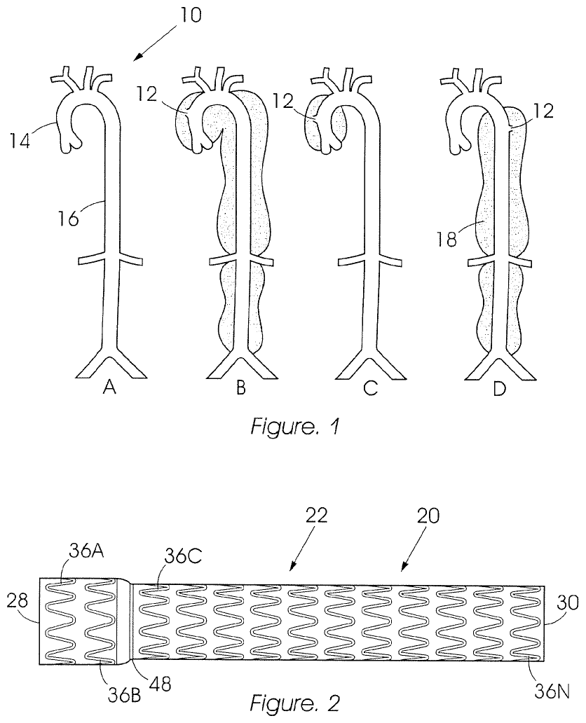

The first illustration of the series in FIG. 1 (i.e. FIG. 1A) depicts the basic anatomy of an aorta 10.

The last three illustration of the series (FIGS. 1B, 1C and 1D) each illustrate the location of an initial tear 12 which will determine the classification of the aortic dissection. For example, a tear in the ascending aorta 14 will cause a Type A dissection (see FIGS. 1B and 1C) whilst a tear in the descending aorta 16 will cause a Type B dissection (see FIG. 1D). Such a tear may extend the length of the aorta as illustrated. An initial tear will allow blood to enter the wall of the aorta. This can create a false lumen 18.

An aortic dissection may be treated with an endovascular stent-graft.

Deploying a stent-graft into the aorta, to occlude the intimal tear, will provide reinforcement to the intimal tear area, and prevent further blood flow through the tear into the false lumen.

However, deploying a conventional stent-graft, designed for aneurysms, comes with the attend problem mentioned above.

FIG. 2 of the accompanying drawings illustrates an endovascular stent graft 20 in accordance with the invention, designed and configured for specifically treating a dissection in the descending aorta.

The stent-graft 20 has a generally tubular body 22 having a tubular wall 24, defining a lumen 26. In the longitudinal direction, the body extends between a proximal end 28 and a distal end 30.

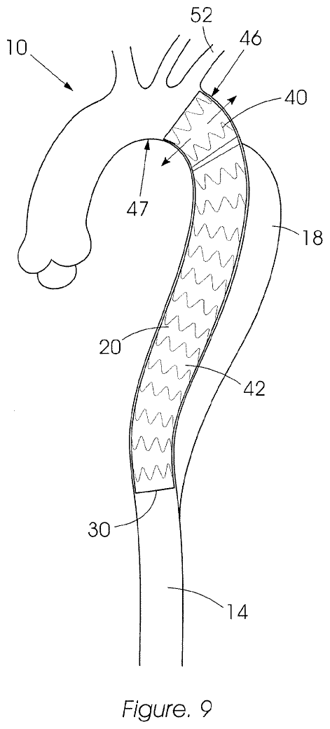

In a first aspect of the invention, the wall 24 of stent graft body 22 is comprised of an inner or support layer 32 and an outer or coating layer 34. This layered structure of the wall is illustrated in FIG. 9.

The inner layer 32 of the tubular wall 24 is made of a knitted or a woven comprising non-elastomeric yarn or a combination of elastomeric and non-elastomeric yarns in any suitable quantity or density to confer to the stent graft body 22 a radial compliance of between 5% and 20% (FIG. 6 illustrates the knitted configuration of this fabric).

In one example, the fabric is a knitted fabric. The preferred yarn is made of a non-elastomeric material such as a polyester material which may be, for example, PET. A knitted mesh of a nonabsorbable, biocompatible material, such as PET, provides supportive frame onto which the coating layer can be applied, whilst being compliant and stretchable. Although the yarn, in this example, may be a non-elastomeric material, the radial compliance inherent in the fabric is achieved by the knit of the yarns.

In another example, the fabric is a woven fabric. In this case, it is preferred that the yarn-type is mixed and includes both elastomeric and non-elastomeric yarns.

Due to the porosity of the fabric, in particular the knitted fabric, which is best illustrated in FIG. 6, the tubular wall 24 must be made blood-tight to prevent blood leakage from the lumen 26. This is done by providing a liquid-tight outer layer 34. Polyurethane is the chosen material for this outer layer due to its elastomeric liquid-tight properties, enabling the outer layer to radially expand with the radial compliance of the inner layer. Furthermore, this polymer can be provided in solid preform, fitted or layered over the inner layer, and heat assisted adherence or chemically bonded thereon. Or, the polymer can be provided in a liquid form, applied to the inner layer by spray coating, dip coating or any other suitable method.

To keep the tubular form of the tubular body, a plurality of circumferential self-expanding stents, respectively designated 36A, 36B, 36C . . . 36N, are engaged to the tubular body 22, spaced at longitudinal intervals.

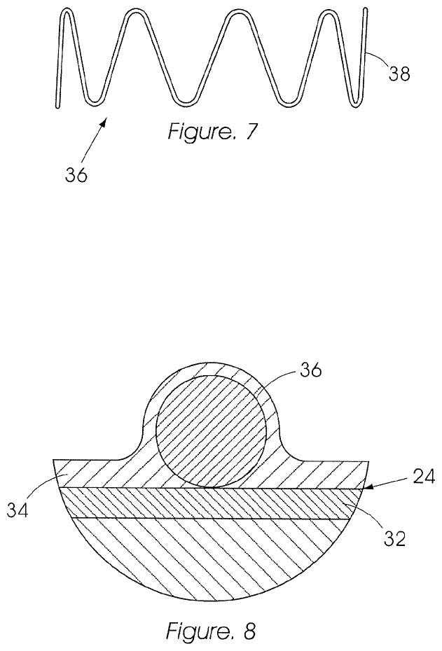

Each stent 36, in this example, has a waveform (zig-zag) wire body 38 of a suitable super-elastic material, such as nitinol, formed with a plurality of peaks 39.1 and a plurality of troughs 39.2. The configuration of a stent is best illustrated in FIGS. 7 and 8.

As illustrated in FIG. 9, each stent is engaged with the tubular body 22 of the stent graft 20 by engagement of the stents to the inner layer 32, prior to the overlay of the outer layer 34, and then fitting, layering or coating the inner layer with the polyurethane to provide the second layer.

Alternatively, employing a coating method, the inner layer is coated with a first coat of polyurethane, the stents are then engaged with or within the first coat, and then a second coat polyurethane is applied (both coats constituting the second layer) to encapsulate the stents within the second layer.

Employing both methods, the stents are effectively sandwiched (or partially enclosed) between the layers, or within the outer layer, as illustrated in FIG. 9. Prior to coating, each stent can be pre-attached to the inner layer by adherence, using a suitable adhesive, or by stitching.

In a second aspect of the invention the tubular body 22 is functionally segmented into a proximal “anchor” section 40, of cylindrical form, a distal “treatment” section 42, of tapered form, and a transition section 44 which tapers the tubular body from the anchor section to the treatment section.

The treatment section is elongate, with a length between 50 mm and 250 mm. This length, ultimately, is based on the pathology of the aorta from the level of the left subclavian artery and distal along the descending thoracic aorta based on the location of the dissection. These centre line distances can be as short as 50 mm to 150 mm for acute traumatic dissection to 250 mm and above for chronic type B dissections. The length of the anchor section and the transition section, combined, is relatively short at, say, approximately 30 mm. This length is the clinical distance from the distal ostia of the common carotid, across the distal traverse aortic arch to the distal ostium of the left subclavian artery.

In a preformed configuration, prior to stent engagement, the tubular body is sized to approximate the profile of a native pre-dissected aorta with:
the anchor section, in a vessel deployed configuration, having a first diameter which is sized to approximate the aorta at the proximal landing zone 46, adjacent or within the aortic arch; and
the treatment section having a straight taper, from a third diameter at an end 48 adjacent the transition section to a fourth diameter at the distal end 30, which is sized to approximate the aorta as it tapers away from the aortic arch 47.

The third diameter corresponds to the aortic diameter distal to the left subclavian. The aorta tapers naturally from the aortic root, through the arch and descending thoracic aorta. The aorta presents approximately a 1 mm tapering from the distal traverse aortic arch (proximal to the left subclavian artery) to the descending thoracic aorta. Thus the third diameter corresponds to the diameter at the level of the aorta distal to the left subclavian artery.

The fourth diameter is the distal end of the device. The descending thoracic aorta tapers approximately 1 mm in diameter per 150 mm of longitudinal length. Thus, for a stent graft which is approximately 200 to 250 mm in length, it will taper approximately 2 mm from the third diameter to the forth diameter following the natural taper.

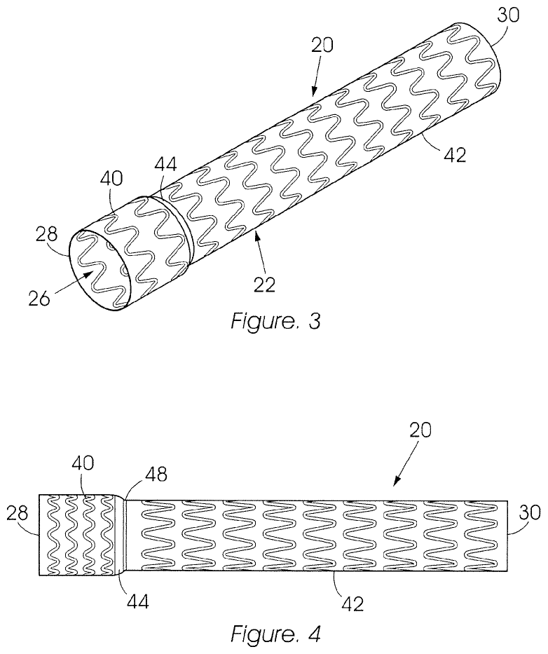

A plurality of stents 36 of a first set and a second set are engaged with the anchor section 40 and the treatment section 42 respectively. Different embodiments of the invention may have different number of stents. For example, in FIG. 3, the anchor section has two associated stents, in FIG. 4, there are four. Along the treatment section, preferably, there is a stent for each 13 mm to 15 mm of length. Notwithstanding the numerical variance, the stents of the first and the second set are of differing construct, wherein:
the stents of the first set are adapted to bias the anchor section from the first diameter of the vessel deployed configuration to a second diameter of a free standing configuration;
the stents of the second set are adapted to maintain the tapered form of the treatment segment (irrespective of the position of the anchor section) without imposing a radial force at or beyond the diameter of the treatment segment or the of the aorta, in its native pre-dissected condition.

In a particular example, first, third and fourth diameters can be 28 mm, 27 mm and 26 mm respectively (however, these diameters will change on the stent-graft in accordance with the specific aortic architecture of the patient being treated which depends upon factors such as age, gender, race and ethnicity), with the second diameter exceeding the first diameter by between 5% and 20%, but typically this difference is about 10% i.e. +/−31 mm.

In this aspect of the invention, the wall 24 of stent graft body 22 may not have two layers, merely comprised of an inner (first) layer 32. In this example, the first layer typically is composed of a woven fabric including a mix of elastomeric and non-elastomeric yarns. Being less porous, and including an elastomeric element within the fabric, the outer (second) liquid-tight layer 34 is not always necessary.

In another example of this aspect, the wall 24 does include a first and a second layer (32, 34), with the first layer typically being a knitted fabric, over which the liquid-tight elastomeric second layer is placed.

In either example, the fabric making up the first layer is sufficiently compliant to accommodate the radial expansion from the first diameter to the second diameter. The advantage of preforming the anchor section 40 of the tubular body 22 with a first diameter which is smaller than a second diameter is that, on deployment which necessitates diametric contraction of the anchor section towards to the first diameter, the tubular wall 24 will not pleat or infold but retain its cylindrical shape. Pleating of the wall will open multiple outlets to blood flow from the lumen of the stent-graft.

The stents 36 of the first set are oversized to achieve the about 10% expansion, relatively to the preformed dimensions of the anchor section 40. These stents also are adapted to compress circumferentially to at least the first diameter when required in deployment of the stent-graft, and to expand circumferentially with inherent spring bias to the second diameter.

In contrast, each stent 36 of the second set is not oversized relatively to the diameter of the part of the treatment section 42 to which it is engaged. These stents will, as a consequence, provide support for the preformed treatment section and will maintain its tapered shaped and dimension. These second set stents can compress and expand diametrically, as with their first set counterparts. However significant diametric change is not necessary as deployment of the stent-graft 20 does not require significant compression or expansion to conform to the dimensions of the preformed treatment section, complementarily shaped as it is to the native aorta into which it is deployed.

That said, the diameter of the treatment section 42 will change, as will the associated stents 36, expanding and recoiling with increasing blood pressure during systolic up-cycle and reducing blood pressure during the diastolic down-cycle. At the diastolic through, the treatment section will retract to a native diameter of the aorta. This ability of the treatment section of the graft to comply with natural diametric changes during a cardiac cycle, due to the inherent compliance of the material making up the wall 24 of stent graft body 22 is best described as the ability to maintain the “windkessel function” of the aorta.

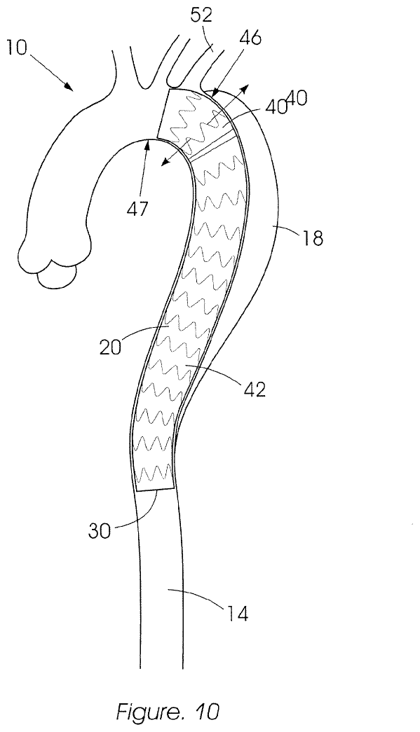

The stent-graft may be introduced into the aorta 10 of the patient being treated with a proprietary introducer device or introducer device known in the art (neither of which are shown). When introduced, the anchor section 40, being oversized by the bias of the stents 36 of the first set, will be compressed from the free standing configuration to the vessel deployed configuration. Once the stent-graft 20 is optimally located, as illustrated in FIG. 9 or 10—with the anchor section 40 in the proximal landing zone 46 such that a part of anchor section 40, the treatment section 42 or both occludes the intimal tear area to prevent further blood flow through the tear into the false lumen 18—the anchor section can be released from compression. The anchor section will expand under action of the stents into load bearing contact with the aortic wall of the landing zone to anchor the stent-graft. The radial force exerted by the anchor section on the aortic wall is illustrated with bi-directional arrows in FIGS. 9 and 10.

In contrast, the treatment section 42 of the stent-graft will have a less aggressive action on the aortic wall of the descending aorta into which it is deployed. At most, the associated second set stents 36 will push the aorta, if partially collapsed due to the dissection, to its native pre-dissection diameter. Pushed back to the native diameter, the treatment section will have negligible load bearing contact with the aortic wall thereafter. With no radial stiffness, so prevalent with prior art stent-grafts, the treatment section is gently compliant with the blood flow pressure waves of each pulse.

Now deployed, the stent-graft 20 is positioned to direct the flow of blood through the lumen 26, bypassing the false lumen 18.

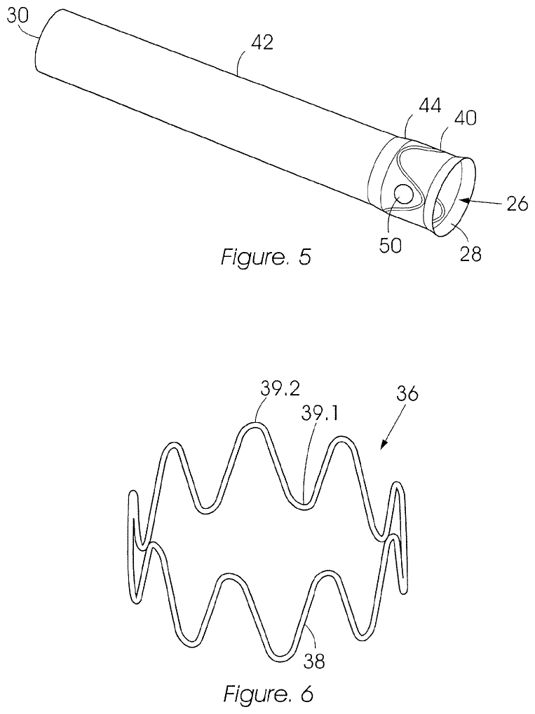

FIG. 5 illustrates a second embodiment of the stent-graft 20A of the invention. This embodiment of the invention has an aperture or fenestration 50 penetrating the tubular wall 24 of the anchor section 40 of the tubular body 22. This embodiment will be put to use when the stent-graft is required to be deployed higher up on the descending aorta 16 due to the higher position of the intimal tear 12. This higher positioning is illustrated in FIG. 10, showing that the landing zone 46 for the stent-graft is located within the aortic arch 47. In the absence of the fenestration, the left subclavian artery 52 will be occluded. With the fenestration, on deployment, the surgeon will ensure that the fenestration is positioned in register with the opening to this artery to provide a blood-flow conduit.

Due to the compliant material of composition, and the lack of over-sized stents in the treatment section 42, the stent-graft 20 of the invention is more compliant/less stiff and with cumulatively lower radial forces imposed on the aorta when compared with stent-grafts of the prior art.

1. (canceled)

2. (canceled)

3. (canceled)

4. (canceled)

5. (canceled)

6. (canceled)

7. (canceled)

8. (canceled)

9. A vascular stent-graft which includes a tubular body having a tubular wall which is comprised of at least a first layer of a fabric, said body including a proximal anchor section of a cylindrical form, preformed with a first diameter and a first length, a distal treatment section of a tapered form and having a second length, and a transition section which tapers the tubular body from the anchor segment to the treatment segment, at least one stent of a first set engaged with the anchor section, adapted to bias the anchor section from the first diameter to a second diameter which exceeds the first diameter.

10. The vascular stent-graft according to claim 9 wherein the first diameter is sized to approximate the aortic diameter at the proximal landing zone.

11. The vascular stent-graft according to claim 9 wherein the first diameter is in a range 18 mm to 40 mm.

12. The vascular stent-graft according to claim 9 wherein the second diameter exceeds the first diameter between 5% and 20%.

13. The vascular stent-graft according to claim 9, wherein the treatment section tapers from a third diameter adjacent the transition section to a fourth diameter at the distal end.

14. The vascular stent-graft according to claim 9, wherein the third diameter is in a range 17 mm to 39 mm and wherein the fourth diameter respectively ranges between 15 mm to 38 mm.

15. The vascular stent-graft according to claim 9 wherein the first length is in a range 20 mm to 50 mm.

16. (canceled)

17. The vascular stent-graft according to claim 9, wherein the second length is in a range 50 to 250 mm.

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. The vascular stent-graft according to claim 9 which includes a plurality of stents of a second set engaged with the treatment section, each adapted to maintain the tapered form.

37. The vascular stent-graft according to claim 36, wherein the tubular wall includes a second layer which covers, coats or encapsulates the first layer

38. The vascular stent-graft according to claim 37, wherein the second layer comprises a liquid-tight material.

39. The vascular stent-graft according to claim 38, wherein the liquid-tight material is polyurethane.

40. The vascular stent-graft according to claim 38, wherein the liquid-tight material is provided as a sheet or a tube, which covers and binds to the first layer.

41. The vascular stent-graft according to claim 38, wherein the liquid-tight material coats the first layer.

42. The vascular stent-graft according to claim 37, wherein the stents of the first set and the stents of the second set are fixed to the anchor section and the treatment section respectively, interposed between the first layer and the second layer or encapsulated within the second layer.

43. The vascular stent-graft according to claim 36, wherein each stent of the first set and the second set is a circumferential self-expanding stent.

44. The vascular stent-graft according to claim 43, wherein the anchor section has a plurality of stents of the first set engaged therewith.

45. The vascular stent-graft according to claim 43, wherein each stent of the second set is engaged with the treatment section sequentially along the second length.

46. The vascular stent graft according to claim 43, wherein each stent of the first set is adapted to maintain the second diameter against an elastic recoil force imposed by the aorta on the anchor section.

47. The vascular stent-graft according to claim 43, wherein each stent of the second set is adapted to maintain the tapered form of the treatment segment.
